(19) United States
(12) Patent Application Publication
(10) Pub. No.: US 2022/0134066 A1
(43) Pub. Date: May 5, 2022

(54) SUBCUTANEOUS DRAIN WITH VALVE

(71) Applicants: Ezriel Kornel, Bedford, NY (US); Michael R. Bielski, Manorville, NY (US); Donald L. Hannula, San Luis Obispo, CA (US)

(72) Inventors: Ezriel Kornel, Bedford, NY (US); Michael R. Bielski, Manorville, NY (US); Donald L. Hannula, San Luis Obispo, CA (US)

(21) Appl. No.: 17/516,080

(22) Filed: Nov. 1, 2021

Related U.S. Application Data
(60) Provisional application No. 63/107,748, filed on Oct. 30, 2020.

(51) Int. Cl. A61M 27/00; A61M 39/02

(52) U.S. Cl. CPC A61M 27/00; A61M 2039/062; A61M 2210/0693; A61M 39/0208

(57) ABSTRACT

A subcutaneous drain includes cap portion and a base with a central opening including a valve element that opens and closes to allow for withdrawal and addition of fluid into the patient's body. In embodiments, the drain may include a seal assembly to prevent leakage around a drain catheter that passes through the drain.

### CROSS-REFERENCE TO RELATED APPLICATIONS

The present application claims benefit of and priority to U.S. Provisional Patent Application Ser. No. 63/107,748 entitled SUBCUTANEOUS DRAIN WITH VALVE, filed Oct. 30, 2020, the entire content of which is hereby incorporated by reference herein.

### BACKGROUND

### Field of the Disclosure

The present disclosure is generally related to a subcutaneous drain including a valve that is selectively opened and closed. The subcutaneous drain may include a seal element to prevent leakage around a drain catheter when the drain catheter is inserted through and received in the valve.

### Related Art

While there are a variety of known subdural drains, the prior art subdural drain devices suffer from one or more disadvantages. Conventional drains in the prior art allow for selective removal of drainage catheters or lumens, however, this removal may allow for leakage of fluid from the subdural space through the passageway that the drainage catheter or lumen passes through the drain.

Accordingly, it would be desirable to provide a non-invasive diagnostic assembly that avoids these and other problems.

### SUMMARY

In light of the above, an improved subcutaneous drain is disclosed.

The present disclosure is generally related to an improved subdural drain that includes a valve to prevent drainage when a draining catheter is not in place in the drain. In embodiments, the subdural drain may also include a seal element to prevent leakage around the draining catheter when it is in place.

A subcutaneous drain in accordance with an embodiment of the present application includes a base configured for contact with a user's body, wherein the base includes: a first portion extending horizontally outward and including a bottom surface in contact with the patient's body; a second portion extending downward from the first portion and into the user's body; and a cap mounted in the base; wherein the cap includes: a semispherical top portion extending upward from the base; and a cylindrical bottom portion extending downward into the user's body and positioned within the second portion of the base; and a central opening extending through the semispherical portion and in fluid communication with the cylindrical bottom portion; and a valve mounted in the central opening and movable between an open position and a closed position.

In embodiments, the valve is a slit valve including: a first flexible element; and a second flexible element, wherein the first flexible element and the second flexible element are movable relative to each other from the closed position into the open position when pressure is applied in the first direction.

In embodiments, the first flexible element and the second flexible element are biased into the closed position.

In embodiments, the first flexible element and the second flexible element are configured to remain in the closed position when pressure is applied in a second direction, opposite the first direction.

In embodiments, the valve is a cruciform valve.

In embodiments, the semispherical portion includes an outer wall element; and an inner wall element spaced radially inward relative to the outer wall element.

In embodiments, the outer wall element is made of a flexible, resilient material.

In embodiments, the inner wall element is made of a flexible, resilient material.

In embodiments, the outer wall and the inner wall are positioned to cover a space between the lower portion of the base and the cylindrical cap portion.

In embodiments the valve includes a slot formed in the semispherical top portion of the cap and configured to receive a catheter.

### BRIEF DESCRIPTION OF THE DRAWINGS

Exemplary embodiments of the present disclosure will be described with reference to the accompanying figures, wherein:

FIG. 1 illustrates a top perspective view of a subcutaneous drain including a valve in accordance with an embodiment of the present disclosure;

FIG. 1A illustrates a perspective view of a subcutaneous drain with a catheter passing therethrough in accordance with an embodiment of the present disclosure;

FIG. 2 is a cross-sectional view of the subcutaneous drain of FIG. 1 in accordance with an embodiment of the present disclosure;

FIG. 2A is a top perspective view of the subcutaneous drain of FIG. 1 including a cruciform slit in the valve in accordance with an embodiment of the present disclosure;

FIG. 3 illustrates a top perspective view of a subcutaneous drain including a seal assembly in accordance with another embodiment of the present disclosure;

FIG. 4 is a cross-sectional view of the subcutaneous drain of FIG. 3 in accordance with an embodiment of the present disclosure;

FIG. 5 is a cross-sectional view of a subcutaneous drain including a valve and a seal assembly in accordance with another embodiment of the present disclosure;

FIG. 6 is a cross-sectional view of the subcutaneous drain of FIGS. 1-2 with a hypodermic needle piercing a portion thereof in accordance with an embodiment of the present disclosure;

FIG. 7 is a cross-sectional view of the subcutaneous drain of FIGS. 1-2 with a drain catheter extending therethrough in accordance with an embodiment of the present disclosure;

FIG. 7A is a top view of the subcutaneous drain of FIGS. 1-2 with the drain catheter extending through the open valve in accordance with an embodiment of the present disclosure;

FIG. 8 is a cross sectional view of the subcutaneous drain of FIGS. 3-4 with a drain catheter extending therethrough in accordance with an embodiment of the present disclosure; and

FIG. 9 is a cross-sectional view of the subcutaneous drain of FIG. 5 with a drain catheter extending therethrough in accordance with an embodiment of the present disclosure.

### DETAILED DESCRIPTION

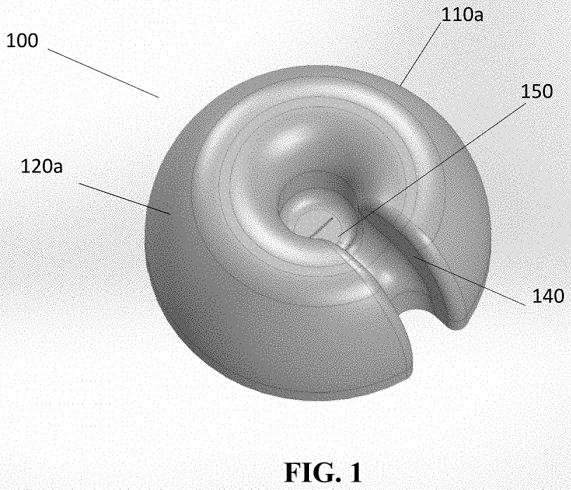

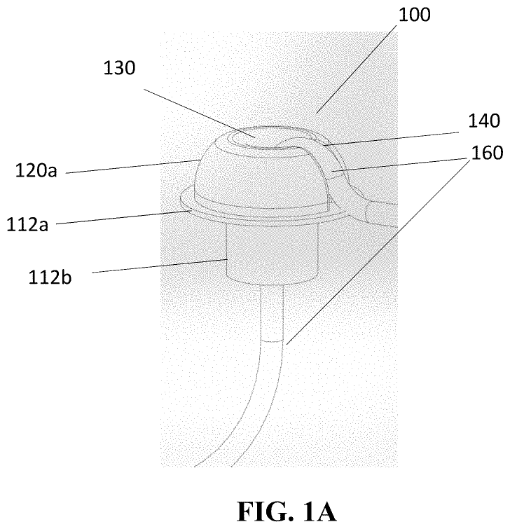

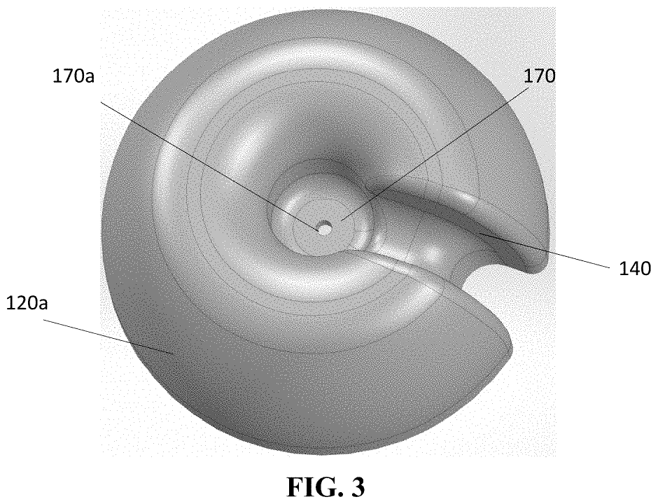

The present disclosure is generally related to an improved subcutaneous drain 100 that includes a valve element 150 to prevent drainage when a drain tube or catheter 160 (see FIG. 1A, for example) is not present in the drain. In embodiments, a seal assembly 170 (see FIG. 3, for example) may be provided to prevent leakage of fluid around the drain tube or catheter 160 when it is in place.

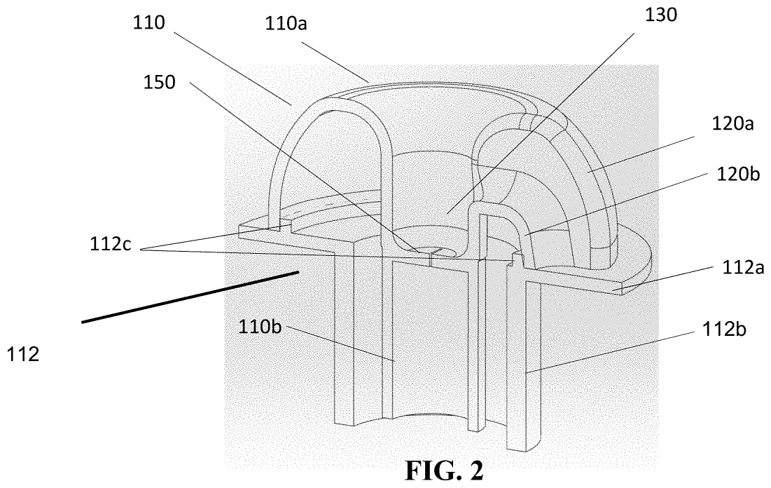

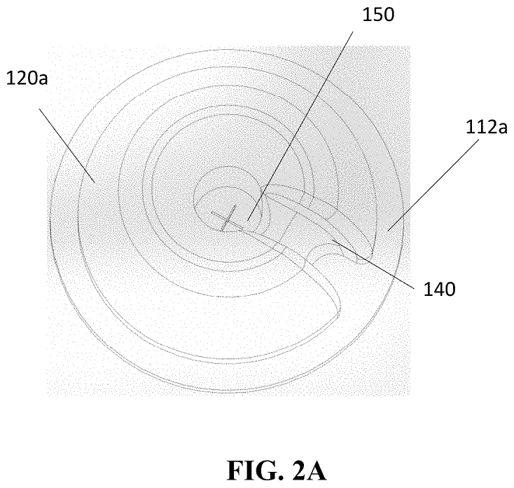

FIG. 1 illustrates an embodiment of a subcutaneous drain 100 in accordance with an embodiment of the present disclosure. In embodiments, the subcutaneous drain 100 is intended for use in a patient's skull, however, the drain may be used in other applications and other body parts, if desired. In embodiments, the drain 100 may be used to drain fluid from any cavity or portion of a patient's body, including the peritoneum or pleura, for example. In embodiments, the subcutaneous drain 100 includes a cap 110 (see FIG. 2, for example) mounted on a base 112 (see FIG. 2, for example). In embodiments, the cap 110 has a mushroom shape with a semispherical top portion 110a and a cylindrical stem portion 110b (see FIG. 2, for example). In embodiments, the top portion 110a has a smooth outer wall portion 120a and an inner wall portion 120b spaced inward from the outer wall 120a. In embodiments, the size of the drain 100 may depend on a size of a burr hole that may be provided in the patient's skull to accommodate the drain 100. In embodiments, the size of the drain 100 may be minimized such that the size of the burr hole may be minimized, however, the size of the drain may vary. In embodiments, the outer and inner wall portions 120a, 120b may be made of a flexible and resilient material such that they can be pushed inward and return to their original shape.

In embodiments, the base 112 includes an upper flange assembly 112a above which the outer wall portion 120a and inner wall portion 120b extend. In embodiments, an inner lip 112c may be provided inside the inner wall portion 120b which may be used to align the components to ensure consistency. In embodiments, the flange assembly 112a extends horizontally out beyond the outer wall portion 120a. In embodiments, the bottom surface of the flange assembly 112a will contact a user's skull, or other part of the user's body, when the drain 100 is positioned in the user's skull and under their skin. In embodiments, where the drain 100 is used with respect to another part of the user's body, the bottom surface of the flange assembly 112a contact's the user's body. In embodiments, the flange 112a may be secured to the user's skull, or another part of the user's body in any suitable manner such as using sutures, staples, adhesives or glue or any other clinically acceptable means of attachment. The lower hollow cylindrical protrusion 112b extends downward from a center of the upper flange assembly 112a. In embodiments, the stem portion 110b of the cap 110 is positioned within the cylindrical protrusion 112b with a lateral space provided between them. In embodiments, the minimum space between the stem portion 110b and the protrusion 112b is 1 mm (0.039 inches). In embodiments, the spacing between the stem portion 110b and the protrusion 112b may vary. Reasons for the variation of spacing may include: 1) changing the dimensions of the central opening based upon the drainage catheter diameter that is clinically required or preferred; 2) varying the lateral space that is in fluid communication with the subdural space to allow for more or less flow of fluid into the lateral space or cap. In embodiments, the protrusion 112b is substantially cylindrical, however, may taper inward toward the bottom to allow for easier placement of the drain 100 in the burr hole in the user's skull or other part of the users body. In embodiments, a burr hole is provided in the user's skull to allow insertion of the drain 100, and specifically, the protrusion 112b with the stem 110b positioned therein. In embodiments, where the drain 100 may be used in another part of the user's body, the protrusion 112b and stem 110b may be positioned in another part of the user's body.

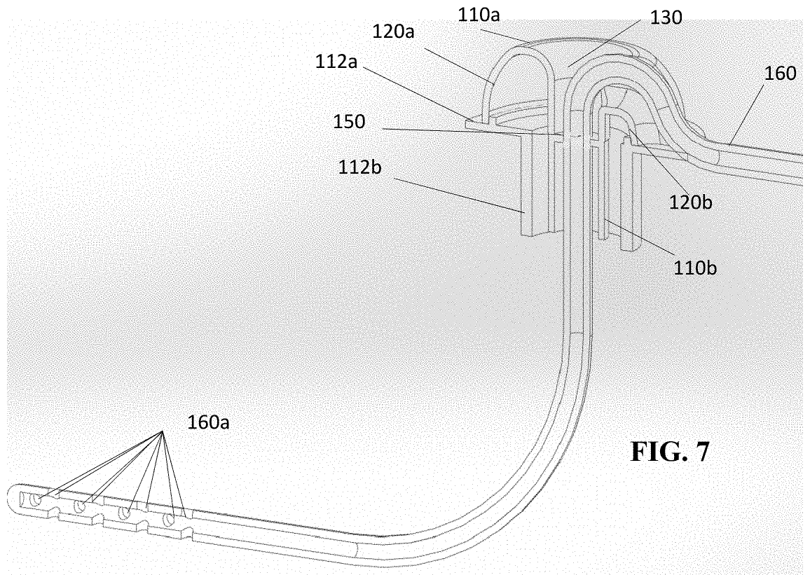

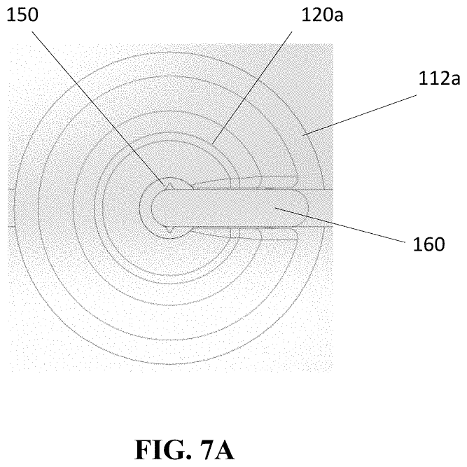

In embodiments, a central opening 130 is provided in the top portion 110a of the cap 110 and extends into the stem 110b to provide for fluid communication between the top of the cap 110 and the user's cranium, or other body part, when the drain 100 is in place in the user's skull, or other body part. In embodiments, a side slot 140 (see FIG. 1, for example) may be provided in the cap portion 110a extending radially outward from the central opening 130 through the outer and inner wall portions 120a, 120b to provide access to the central opening 130 from the side of the cap 110. In embodiments, this side slot 140 may accommodate a drain tube or catheter 160 that may be provided to pass through the central opening 130 and into the patient's cranium, or other body part (see FIG. 7 for example). In embodiments, the catheter 160 is guided in the slot 140 to the central opening 130 to provide kink resistance as can be seen in FIG. 1A, for example. A kink in the catheter 160 would present significant problems since the drain 100 is positioned under the user's skin and the catheter passes through and under the user's skin making it difficult to access the catheter to fix a kink should one form. In embodiments, using the slot 140 to guide the catheter 160 into the central opening 130 helps prevent kinks from forming in the catheter. In embodiments, a proximal end of the catheter 160 extends outside of the user's scalp.

In embodiments, a valve 150 may be provided in the central opening 130 to control flow of fluid into and out of the cranium, or other body part of the patient. As can be seen in FIGS. 1 and 2, a slit valve 150 (see FIG. 2, for example) may be provided in the opening 130. As illustrated in FIGS. 1 and 2, in embodiments, the slit valve 150 may be biased into a closed position to prevent the flow of fluid through the drain 100 either out of or into the user's cranium, or other body part. In embodiments, the slit valve 150 may be made of a flexible and resilient material that is biased in the closed position (see FIG. 2, for example) and allow for the two sides to separate when pressure is applied to the slit in a first direction to move the valve 150 into an open or partially open state to allow for passage of a catheter or lumen to pass therethrough while preventing fluid from exiting when the catheter or lumen is removed. In embodiments, the valve 150 may be structured such that pressure in a second direction, opposite the first direction, does not result in movement of the opposed sides of the valve such that the valve does not open in response to pressure in the second direction and prevents fluid from leaking out of the subcutaneous space. In embodiments, the catheter 160 may be used to apply downward pressure to the slit valve 150 which opens to allow the catheter to pass therethrough into the cranium, or other body part of the patient. In embodiments, the distal end of the catheter 160 may include one or more lateral openings 160a formed in a sidewall thereof through which fluid may enter the catheter from the user's body. In embodiments, fluid from the cranium may flow into the catheter 160 which passes through the valve 150 and drain 100 out of the cranium of the user via the catheter when suction is applied on a proximal end of the catheter. In embodiments, fluid from the cranium may flow into the catheter 160 and out through the valve 150 and drain 100 into an external ventricular drainage (EVD) bag for collection of the fluid without the need to apply suction. In embodiments, the proximal end of the catheter 160 may be positioned outside of the scalp or skin portion of the user such that it can be easily connected to a source of suction and/or an external ventricular drainage (EVD) bag. While the EVD bag is specifically discussed herein, in embodiments, the catheter 160 may be connected to, and fluid may be collected in, any sterile external drainage bag or container such as a closed suction drain with a bulb or any other negative pressure drain system. Any medical drainage system where temporary placement of a drainage catheter that might require aspiration following the removal of the drainage catheter would be suitable for this invention. In embodiments, the catheter 160 may also be used to provide fluids, such as medicine, for example, to the user's cranium. In embodiments, the valve 150 may be a cruciform slit valve (See FIG. 2A, for example) including two slits formed is a cross configuration such that when pressure is applied to the center of the cross configuration, the four elements flex such that the valve is in an open or partially open state. FIG. 2A illustrates an exemplary embodiment of the drain 100 including a valve 150 with a cruciform slit.

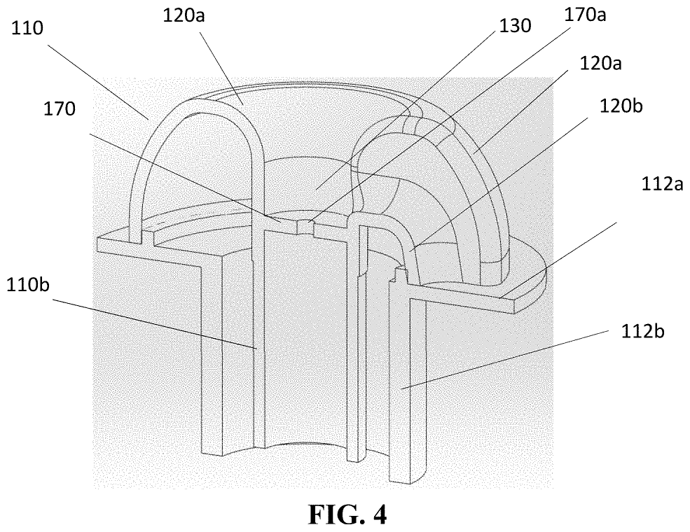

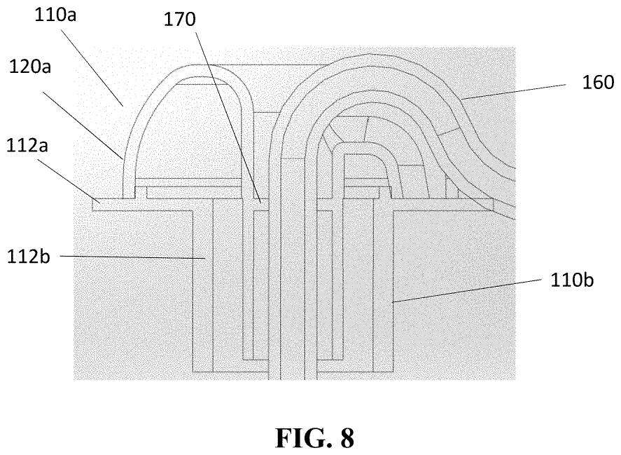

In embodiments, a seal structure 170 (see FIG. 4, for example) may be provided in the central opening 130 to prevent fluid from leaking around the catheter 160 when it is in place. In embodiments, the seal 170 may have a general disk shape that extends across the opening 130 with an open center 170a formed through which the catheter 160 passes. In embodiments, the diameter of the open center 170a is smaller than that of the catheter 160 and the seal 170 is made of a flexible material that stretches to accommodate passing of the catheter through the open center while maintaining contact with the sides of the catheter to prevent fluid from passing around the catheter. In embodiments, the drain 100 may include the seal 170 and not the valve 150 as illustrated in FIGS. 3 and 4. In this case, the catheter 160 will pass through the seal 170 and into the user's cranium, or other body part, to allow for removal or addition of fluid as can be seen in FIG. 8, for example.

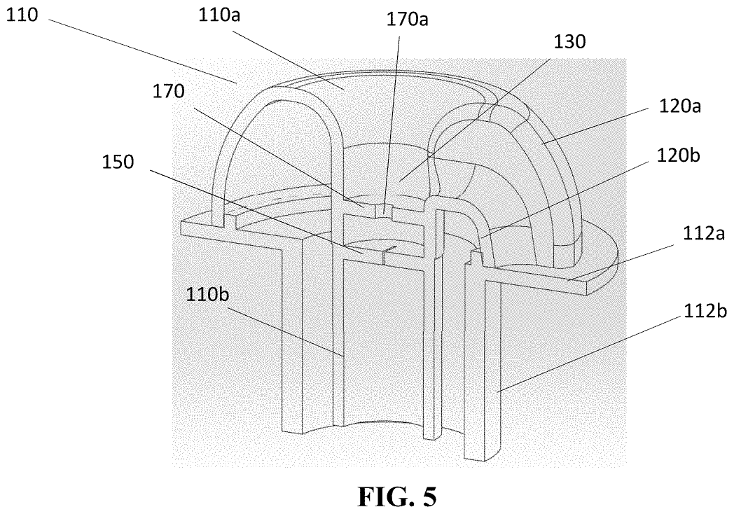

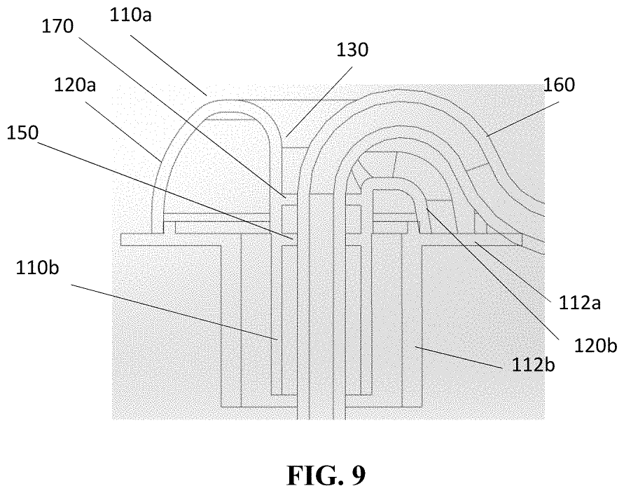

In embodiments, the drain 100 may include both the seal structure 170 and the valve 150 as can be seen in FIGS. 5 and 9, for example. In embodiments, the seal 170 may be positioned above the valve 150 as can be seen in FIGS. 5 and 9. In embodiments, the catheter 160 may pass through seal structure 170 as described above and then through the valve 150 as it is advanced down into the user's cranium. The seal 170 prevents leakage of any fluid that may inadvertently pass through the valve 150 as the catheter 160 passes through it and as fluid is drained or added. As noted above, the catheter 160 may be used to drain fluid from the patient's cranium or to provide fluid, such as medicine, to the patient's cranium, or another body part.

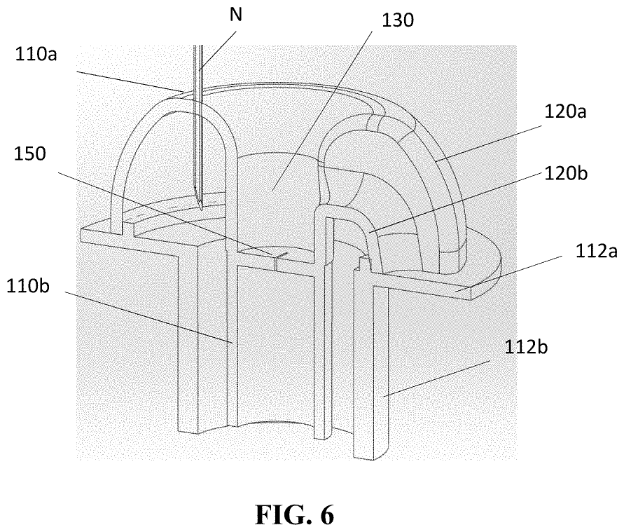

In embodiments, fluid may be provided to, or removed from, the user's cranium, or other body part, through the drain 100 using a hypodermic needle N, for example, as can be seen in FIG. 6. In embodiments, the hypodermic needle N may be inserted through at least the outer wall portion 120a of the cap 110 and down into the lateral space between the stem 110b and the protrusion 112b which is in fluid communication with the patient's cranium. In embodiments, the needle N may be a non-coring needle, for example, a non-coring 25 gage needle, however, other needles may be used. In embodiments, the hypodermic needle N may be used to remove fluid from the user's cranium or to inject fluid, such as medicine, into the user's cranium.

While the drain 100 is discussed herein as being provided in a user's skull and under the user's scalp, or other portion of the user's skin when the drain 100 is used in other applications. In one example, the drain 100 may be used to place a catheter in a lateral ventricle of the brain to drain cerebral spinal fluid (CSF) for example, after trauma that results in bleeding into the ventricle.

While particular embodiments of the present disclosure have been shown and described in detail, it would be obvious to those skilled in the art that various modifications and improvements thereon may be made without departing from the spirit and scope of the disclosure.

What is claimed is:

1. A subcutaneous drain comprising:
a base configured for contact with a user's body,
wherein the base includes:
a first portion extending horizontally outward and including a bottom surface in contact with the patient's body;
a second portion extending downward from the first portion and into the user's body; and
a cap mounted in the base;
wherein the cap includes:
a semispherical top portion extending upward from the base; and
a cylindrical bottom portion extending downward into the user's body and positioned within the second portion of the base; and
a central opening extending through the semispherical portion and in fluid communication with the cylindrical bottom portion; and
a valve mounted in the central opening and movable between an open position and a closed position.

2. The subcutaneous drain of claim 1, wherein the valve is a slit valve comprising:
a first flexible element; and
a second flexible element,
wherein the first flexible element and the second flexible element are movable relative to each other from the closed position into the open position when pressure is applied in the first direction.

3. The subcutaneous drain of claim 2, wherein the first flexible element and the second flexible element are biased into the closed position.

4. The subcutaneous drain of claim 2, wherein the first flexible element and the second flexible element are configured to remain in the closed position when pressure is applied in a second direction, opposite the first direction.

5. The subcutaneous drain of claim 1, wherein the valve is a cruciform valve.

6. The subcutaneous drain of claim 1, wherein the semispherical portion comprises:
an outer wall element; and
an inner wall element spaced radially inward relative to the outer wall element.

7. The subcutaneous drain of claim 6, wherein the outer wall element is made of a flexible, resilient material.

8. The subcutaneous drain of claim 6, wherein the inner wall element is made of a flexible, resilient material.

9. The subcutaneous drain of claim 6, wherein the outer wall and the inner wall are positioned to cover a space between the lower portion of the base and the cylindrical cap portion.

10. The subcutaneous drain of claim 1, further comprising a slot formed in the semispherical top portion of the cap and configured to receive a catheter.
